(19) United States
(12) Patent Application Publication
(10) Pub. No.: US 2020/0069906 A1
(43) Pub. Date: Mar. 5, 2020

(54) SNOW GLOBE HAVING AROMA DIFFUSER FUNCTION

(71) Applicant: RUI&OLIVIA CO., LTD., Seogwipo-si, Jeju-do (KR)

(72) Inventor: Jae Jin CHOI, Seoul (KR)

(21) Appl. No.: 16/490,553

(22) PCT Filed: Sep. 27, 2017
(86) PCT No.: PCT/KR2017/010671

§ 371 Date: Sep. 1, 2019

(30) Foreign Application Priority Data
Mar. 24, 2017 (KR) 20-2017-0001405

(51) Int. Cl. A61M 21/00; G09F 19/00

(52) U.S. Cl. CPC A61M 21/00; A61M 2205/59; A61M 2021/0016; G09F 19/00

(57) ABSTRACT

The present invention relates to a novel snow globe having an aroma diffuser function, which can obtain indoor air freshening effects without installation of a separate aroma diffuser in the room, the snow globe comprising: a glass globe in which a miniature, a transparent liquid, and white flakes or powder are stored; and a support on which the glass globe is placed, wherein all or a part of the support is formed of an absorbent material, and the portion formed of the absorbent material is impregnated with a fragrance oil.

### TECHNICAL FIELD

The following disclosure relates to a snow globe having an aromatic diffuser function, and more particularly, to a snow globe of a new configuration having an aromatic diffuser function capable of obtaining an indoor aromatic effect without separately providing an aromatic diffuser in a room.

### BACKGROUND

A snow globe, also known as a snow ball, refers to a product in which a miniature model, a transparent liquid, or white pieces or powders are placed.

When shaking a snow globe, the pieces or powders mixed in the liquid spread in the glass ball and slowly fall down as if the snow falls.

Since snow globes not only give a romantic and magical uniqueness like snowfall, but also give a decorative function by a miniature model built in a support or glass ball, snow globes are loved as decor accessories.

By the way, snow globes are being utilized as a freshener in addition to a function of simple decorative accessories.

However, since in snow globe products having aromatic functions, a container for filling the freshener needs to be injection-molded, the development cost increases. Also, since electric parts for spraying a flavoring agent filled in a container need to be provided, the manufacturing cost increases.

In addition, when a flavoring agent is sprayed and is used as an interior accessory in a quiet room, generation of noise by rotation of a fan and a motor causes uncomfortable.

### DISCLOSURE

### Technical Problem

Accordingly, the present disclosure provides a snow globe having an aromatic diffuser function, which can overcome spatial and economical limitations occurring when a user purchases both of a snow globe and an aromatic diffuser and places both the snow globe and the aromatic diffuser in a room.

The present invention also provides a new snow globe having economical and practical aromatic diffuser functions, which can obtain an indoor aromatic effect even though a separate aromatic diffuser is not purchased because having a function as an aromatic diffuser while maintaining its inherent function as a snow globe.

### Technical Solution

In one general aspect, a snow globe having an aromatic diffuser function includes: a glass ball storing a miniature model, a transparent liquid, white pieces or powders therein; and a support on which the glass ball is placed, wherein the support is entirely or partially formed of an absorbent material, and a part formed of the absorbent material is impregnated with aromatic oil.

In another general aspect, a snow globe having an aromatic diffuser function includes: a glass ball storing a miniature model, a transparent liquid, white pieces or powders therein; and a support on which the glass ball is placed, wherein the snow globe further includes an aromatic member that has a ring shape and fits to an outer circumferential surface of the support, and the aromatic member is formed of a flexible material and contains aromatic oil.

In another general aspect, a snow globe having an aromatic diffuser function includes: a glass ball storing a miniature model, a transparent liquid, white pieces or powders therein; and a support on which the glass ball is placed, wherein a storage part is formed inside the support to be upwardly opened, and an aromatic oil container is further provided to store aromatic oil and to be received in the storage part.

The support may be provided with an air passage for communicating the storage part to the outside of the support, and the aromatic oil stored in the aromatic oil container may be emitted to the outside of the support through the air passage.

The support may be formed of an absorbent material, and a wick is fitted in the aromatic oil container such that one end thereof is connected to the support, and the aromatic oil stored in the aromatic oil container may be supplied to the support by the wick.

The snow globe may further includes an aromatic member formed of an absorbent material and having a rear surface thereof connected to a wick, wherein: an insertion groove to which the aromatic member is fitted is formed on an outer circumferential surface of the support; a wick insertion hole through which the wick penetrates is formed in the insertion groove to communicate with the storage part; the aromatic member is fitted to the insertion groove such that the wick penetrates the wick insertion hole; and the aromatic oil stored in the aromatic oil container is supplied to the aromatic member through the wick when a front end portion of the wick is inserted into the aromatic oil container.

Other features and aspects will be apparent from the following detailed description, the drawings, and the claims.

### Advantageous Effects

According to an embodiment of the present invention, since the function as an aromatic diffuser is additionally performed while maintaining the original function of the snow globe, consumers do not need to separately purchase an aromatic diffuser to obtain an indoor aromatic effect and to store the aromatic diffuser in a room.

Accordingly, it is economical for consumers to save the cost of purchasing an aromatic diffuser, and since both snow globe and aromatic diffuser are not provided in a room, the space utilization is also excellent.

In particular, in a case where an aromatic member is provided on a support such that an aromatic effect can be obtained by the aromatic member, the support can be formed of various materials. Accordingly, since the selection of material for the support is not limited, the overall appearance of the present invention can be enhanced together with the appearance of the support. Furthermore, when an aromatic oil container is housed in the support and the aromatic member is connected to the aromatic oil container by a wick, aromatic oil stored in the aromatic oil container can be supplied to the aromatic member by the wick. Accordingly, the continuous aromatic effect can be obtained even though aromatic oil is not continuously supplied by a user.

### BRIEF DESCRIPTION OF THE DRAWINGS

FIG. 1 is a perspective view illustrating a snow globe according to a first embodiment of the present invention.

FIG. 2 is a side cross-sectional view illustrating the snow globe according to the first embodiment of the present invention.

FIG. 3 is a perspective view illustrating a snow globe according to a second embodiment of the present invention.

FIG. 4 is a side cross-sectional view illustrating the snow globe according to the second embodiment of the present invention.

FIG. 5 is an exploded perspective view illustrating a support according to a third embodiment of the present invention.

FIG. 6 is a side cross-sectional view illustrating the support according to the third embodiment of the present invention.

FIG. 7 is a cross-sectional view illustrating a snow globe according to a fourth embodiment of the present invention.

FIG. 8 is a perspective view illustrating a snow globe according to a fifth embodiment of the present invention.

FIG. 9 is a cross-sectional view illustrating the snow globe according to the fifth embodiment of the present invention.

### BEST MODE

Hereinafter, exemplary embodiments will be described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

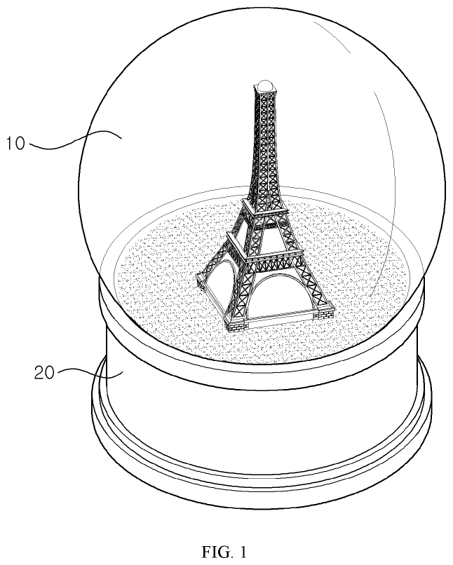

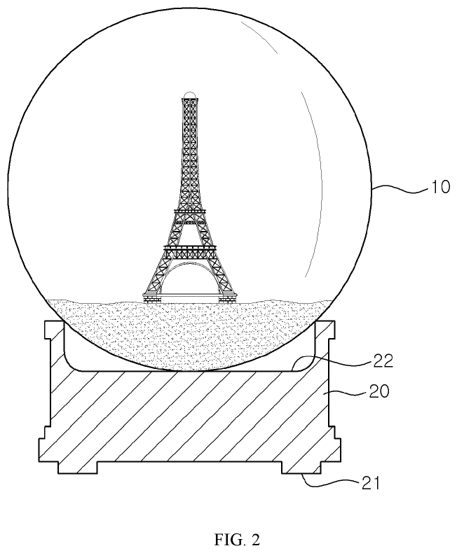

FIG. 1 is a perspective view illustrating a snow globe according to a first embodiment of the present invention, and FIG. 2 is a side cross-sectional view illustrating the snow globe according to the first embodiment of the present invention. As shown in FIGS. 1 and 2, the snow globe includes a glass ball 10 and a support 20 on which is seated.

The glass ball 10 is formed of transparent glass and includes a miniaturized model, a transparent liquid, and white pieces or powders for showing a snowing effect. The glass ball 10 has the same structure as a glass ball used in a related-art snow globe.

Meanwhile, the support 20 has a glass ball 10 placed on an upper surface thereof, and a seating recess 22 on which a glass ball 10 is seated is formed on the upper surface of support 20. The support 20 is formed of an absorbent material so as to have various shapes, and aromatic oil is impregnated. As the absorbent material, gypsum may be used.

The type of aromatic oil that is usable is not particularly limited, and various types of aromatic oils may be used.

As aromatic oil is impregnated into the support 20, the support 20 acts as an aromatic diffuser. In other words, the present invention functions as an aromatic diffuser as well as a snow globe. When the snow globe according to an embodiment of the present invention is disposed in a room, the aromatic oil impregnated in the support 20 is gradually emitted to obtain an indoor aromatic effect. Accordingly, it is not necessary to separately purchase an aromatic diffuser when the snow globe according to an embodiment of the present invention is provided indoors.

The support 20 may be divided into two layers, an upper layer and a lower layer, which are formed of different materials. The upper layer is formed of an absorbent material and corresponds to the upper part of the support 20, which corresponds to a space connecting the outer surface and the bottom surface of the seating recess 22.

That is, the seating recess 22 is formed on the upper surface of the upper layer of the support 20 so as to be depressed in a bottom surface direction, and the impregnated aromatic oil is continuously evaporated through the outer surface of the support 20 and the outer surface of the seating recess 22.

The seating recess 22 serves to support the glass ball 10, and also serves to widen an evaporation area. For this, corrugation may be formed in the seating recess 22 to maximize the evaporation area.

On the other hand, the lower layer corresponds to the lower part of the support 20, and is formed of a non-absorbent material. The lower layer may be a leg 21 supporting the undersurface of the support 20.

The lower layer blocks the aromatic oil impregnated in the upper layer, and prevents the aromatic oil from flowing or leaking out through the undersurface of the support 20.

On the other hand, when all the aromatic oil impregnated into the support 20 is diffused, a user may additionally impregnate the aromatic oil into the support 20. That is, when the aromatic oil is dropped on the support 20 by an eyedropper or the like, the support 20 absorbs the aromatic oil and gradually emits the aromatic oil, such that the aromatic effect can be continuously obtained.

As shown in FIG. 2, the leg 21 is formed on the undersurface of the support 20 such that the undersurface of the support 20 is spaced from the floor. Thus, since the undersurface of the support 20 is spaced away from the floor and thus the support 20 makes relatively larger contact with air, the aromatic effect can be increased.

Corrugation may also be formed on the undersurface of the support 20 to maximize the contact area with air.

It has been described that the whole of the support 20 is formed of an absorbent material and the whole of the support 20 is impregnated with aromatic oil. However, in some cases, only a part of the support 20 may be formed of an absorbent material to be impregnated with the aromatic oil.

### MODE FOR INVENTION

Hereinafter, another embodiment of the present invention will be described, but the description about the same configuration and effects as those of the above-described embodiment will be omitted.

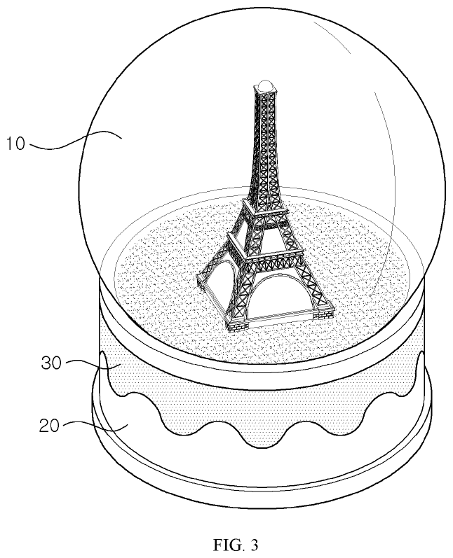

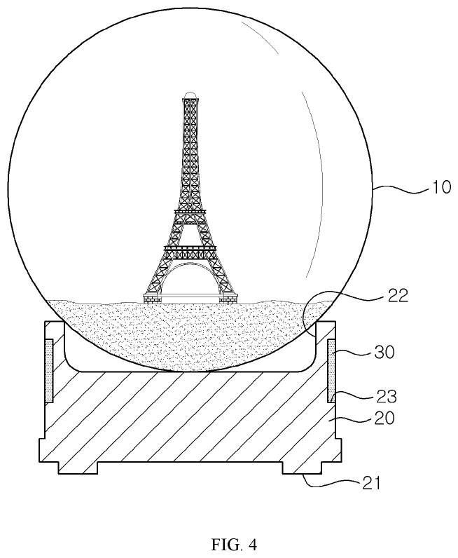

FIG. 3 is a perspective view illustrating a snow globe according to a second embodiment of the present invention, and FIG. 4 is a cross-sectional view illustrating the snow globe according to the second embodiment of the present invention. In this embodiment, the support 20 is provided with a separate aromatic member 30 containing aromatic oil, and the support 20 serves as an aromatic diffuser.

The aromatic member 30 is formed of a flexible material such as soft synthetic resin, silicone, or rubber so as to have a ring shape, and aromatic oil is contained at the time of forming. If necessary, an aromatic oil capsule encapsulating aromatic oil may be mixed with the flexible material to form the aromatic member 30.

This aromatic member 30 is fitted to the outer circumferential surface of the support 20. Since the aromatic member 30 is formed of a flexible material, the state of the aromatic member 30 fitted to the support 20 may be maintained. When the aromatic member 30 is formed in a ring shape having flexibility, the aromatic member 30 can be easily fixed to the support 20 without a separate tool or part for fixing the aromatic member 30 to the support 20. The insertion groove 23 is formed on the outer circumferential surface of the support 20 so as to have a shape corresponding to the aromatic member 30. Thus, when the aromatic member 30 is inserted into the insertion groove 23, the aromatic member 30 does not protrude to the outside. Accordingly, the integrity of the aromatic member 30 and the support 20 is improved and the overall appearance of the support 20 is also improved.

Like this embodiment, when the aromatic member 30 is fitted to the support 20, the aromatic effect can be obtained by the aromatic member 30 and the support 20 can be decorated by the shape and color of the aromatic member 30, and thus the appearance of the support 20 is also improved. In addition, when the aromatic oil contained in the aromatic member 30 is exhausted, only the aromatic member 30 can be replaced with a new one. Also, a user can select different shapes, colors, and types of aromatic member 30. Accordingly, when another aromatic member 30 different from the old aromatic member 30 is selected, it is possible to produce different atmospheres with only one snow globe and to obtain an effect of purchasing a new snow globe.

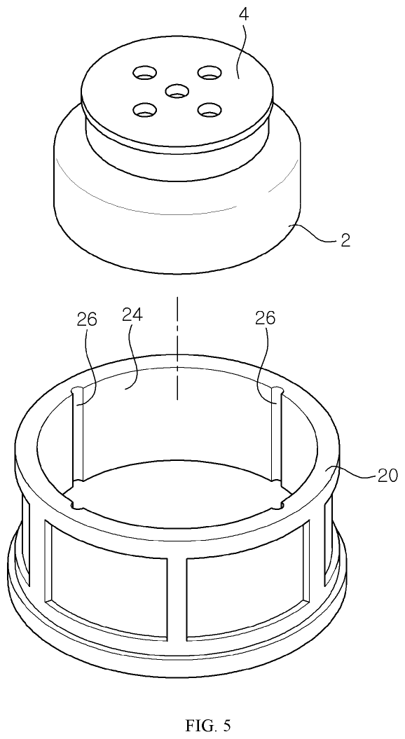

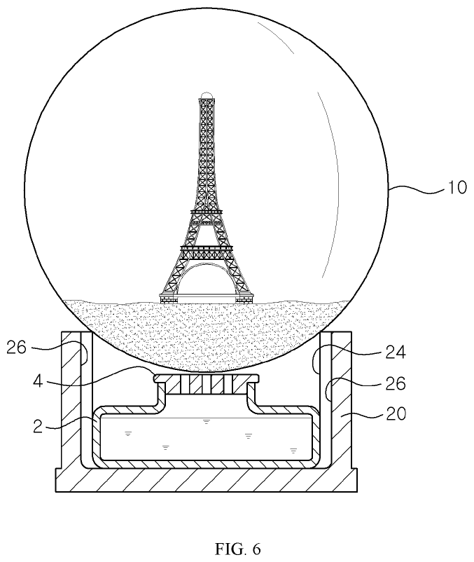

FIG. 5 is an exploded perspective view illustrating a support according to a third embodiment of the present invention, and FIG. 6 is a cross-sectional view illustrating the support according to the third embodiment of the present invention. In this embodiment, a storage part 24 upwardly opened is formed inside the support 20, and an aromatic oil container 2 containing aromatic oil is stored in the storage part 24. Reference numeral 4 denotes a cover of the aromatic oil container 2.

Also, an air passage 26 is formed in the support 20 to communicate the storage part 24 with the outside such that the aromatic oil is emitted to the outside. Accordingly, even when the glass ball 10 is placed on the support 20, the aromatic oil can be emitted to the outside through the air passage 26 to obtain an aromatic effect.

The air passage 26 is shown as being opened to the upper surface of the support 20 along the inner surface of the storage part 24 in this embodiment, but the air passage 26 may be formed in various shapes so as to communicate the storage part 24 with the outside.

In this embodiment, when the aromatic oil stored in the aromatic oil container 2 is exhausted, the aromatic oil container 2 may be replaced with a new one.

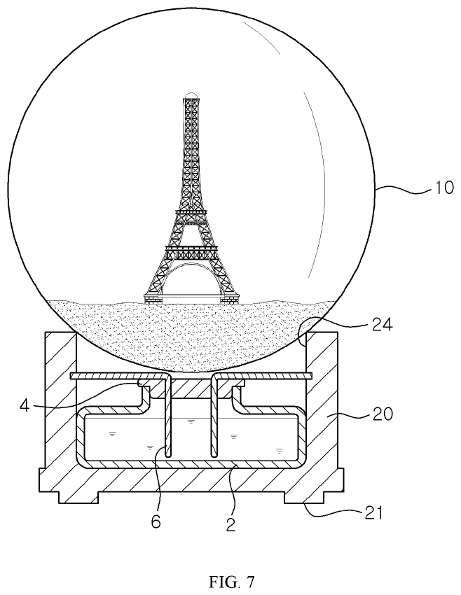

FIG. 7 is a cross-sectional view illustrating a snow globe according to a fourth embodiment of the present invention. In this embodiment, the whole of the support 20 is formed of an absorbent material, and the aromatic oil container 2 and the support 20 are connected to each other by a wick 6. That is, the wick 6 is inserted into the aromatic oil container 2, and the other end of the wick 6 is allowed to make contact with the support 20. Thus, since the aromatic oil stored in the aromatic oil container 2 is supplied to the support 20 by the wick 6, the aromatic effect can be consistently maintained.

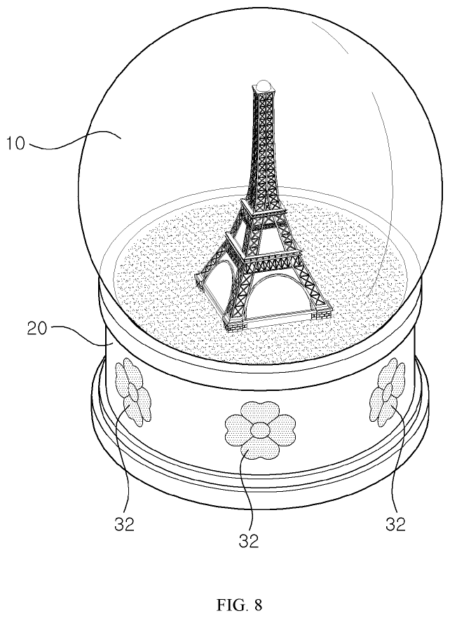

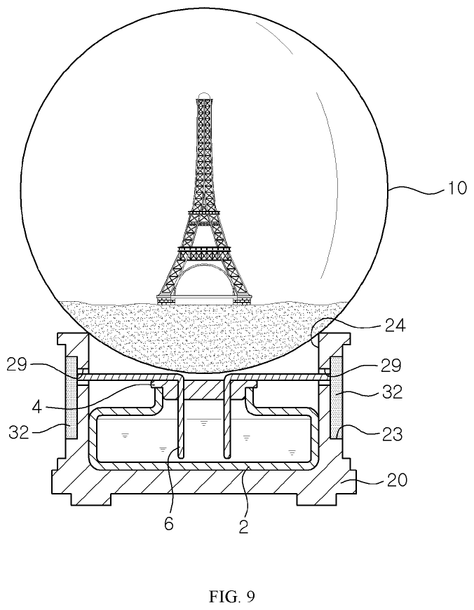

FIG. 8 is a perspective view illustrating a snow globe according to a fifth embodiment of the present invention, and FIG. 9 is a cross-sectional view illustrating the snow globe according to the fifth embodiment of the present invention. In this embodiment, the aromatic member 32 formed of an absorbent material is fitted to the support 20, and the aromatic member 32 is configured to be connected to the aromatic oil container 2 by the wick 6.

In this embodiment, the aromatic member 32 is formed of an absorbent material such as gypsum, wood, and stone, and the wick 6 is connected to the rear surface of the aromatic member 32. A concave groove 23 into which the aromatic member 32 is inserted is formed on the outer circumferential surface of the support 20. Also, a wick insertion hole 29 is formed in the concave groove 23 to communicate with the storage part 24 of the support 20, and the wick 6 penetrates the wick insertion hole 29.

Accordingly, when the aromatic member 32 is fitted to the concave groove 23 such that the wick 6 penetrates the wick insertion hole 29, the front end portion of the wick 6 is located inside the storage part 24 of the support 20, and the front end portion of the wick 6 is fitted into the aromatic oil container 2. Thus, the aromatic oil stored in the aromatic oil container 2 is supplied to the aromatic member 32 by the wick 6 and thus the aromatic effect is consistently maintained. If necessary, the aromatic member 32 may be firmly fixed to the concave groove 23 by an adhesive.

Particularly, in the case where the aromatic member 32 is inserted into the support 20 to obtain an aromatic effect like this embodiment, only the aromatic member 32 is formed of an absorbent material and the material for the support 20 can be variously selected. Also, the support 20 is decorated by the aromatic member 32, and thus the appearance of the support 20 is further improved.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

1. (canceled)

2. (canceled)

3. A snow globe having an aromatic diffuser function, comprising:
a glass ball (10) storing a miniature model, a transparent liquid, white pieces or powders therein;
a support (20) on which the glass ball (10) is placed;
a storage part (24) formed inside the support (20) to be upwardly opened; and
an aromatic oil container (2) storing aromatic oil and received in the storage part (24), wherein
an aromatic member (32) formed of an absorbent material and having a rear surface thereof connected to a wick (6) is further provided;
an insertion groove (23) to which the aromatic member (32) is fitted is formed on an outer circumferential surface of the support (20);
a wick insertion hole (29) through which the wick (6) penetrates is formed in the insertion groove (23) to communicate with the storage part (24);
the aromatic member (32) is fitted to the insertion groove (23) such that the wick (6) penetrates the wick insertion hole (29); and
the aromatic oil stored in the aromatic oil container (2) is supplied to the aromatic member (32) through the wick (6) when a front end portion of the wick (6) is inserted into the aromatic oil container (2).

4. (canceled)

5. (canceled)

6. (canceled)
